(19) United States
(12) Patent Application Publication
(10) Pub. No.: US 2021/0041434 A1
(43) Pub. Date: Feb. 11, 2021

(54) Systems and Methods for Measuring Binding Kinetics of Analytes in Complex Solutions

(71) Applicant: MagArray, Inc., Milpitas, CA (US)

(72) Inventors: Heng Yu, Campbell, CA (US); Sebastian J. Osterfeld, Ann Arbor, MI (US); Kalidip Choudhury, Palo Alto, CA (US)

(21) Appl. No.: 16/985,970

(22) Filed: Aug. 5, 2020

Related U.S. Application Data
(60) Provisional application No. 62/883,515, filed on Aug. 6, 2019.

(51) Int. Cl. G01N 33/557; G01N 33/543

(52) U.S. Cl. CPC G01N 33/557; G01N 33/54326

(57) ABSTRACT

Methods for quantitatively determining a binding kinetic parameter of a molecular binding interaction, for example wherein the determination involves a complex sample, are provided. Aspects of embodiments of the methods include: producing a magnetic sensor device including a complex sample including a magnetic sensor in contact with an assay mixture including a magnetically labeled molecule to produce a detectable molecular binding interaction; obtaining a real-time signal from the magnetic sensor; and quantitatively determining a binding kinetics parameter of the molecular binding interaction from the real-time signal. Also provided are systems and kits configured for use in the methods.

### CROSS-REFERENCE TO RELATED APPLICATIONS

This application claims benefit of priority to U.S. Provisional Application No. 62/883,515, filed Aug. 6, 2019, the disclosure of which is incorporated herein by reference in its entirety.

### INTRODUCTION

Biological processes are dictated by molecular interactions between pairs of first and second molecules. Examples of such molecular interactions include nucleic acid hybridization interactions, protein-protein interactions, protein-nucleic acid interactions, enzyme-substrate interactions and receptor-ligand interactions, e.g., antibody-antigen interactions and receptor-agonist or antagonist interactions. Affinity-based sensing of DNA hybridization, antigen-antibody binding, and DNA-protein interactions have all been shown to play important roles in basic science research, clinical diagnostics, biomolecular engineering, and drug design. As the state of the art advances, demand for accurate, sensitive, high throughput and rapid methods for determination of molecular identities and reaction details place constant pressure on evolving analytical methods. To meet these pressing needs, researchers have turned to molecular labels in order to improve sensitivity for detection of rare molecules. Such labels, however, can alter diffusion and steric phenomena. In addition, high throughput, or speed requirements often prohibit the use of classical equilibrium methods, so that a detailed understanding of reaction kinetics, diffusion phenomena, and the implications of surface immobilization become vital for the extraction of meaningful reaction parameters.

When evaluating the kinetics of a given molecular interaction, various quantitative kinetic parameters may be of interest. One quantitative kinetic parameter of interest is the association rate constant. The association rate constant (i.e., kₐ, kₒₙ) is a mathematical constant describing the bonding affinity of two molecules at equilibrium, such as the bonding affinity of an antibody and an antigen. Another quantitative kinetic parameter of interest is the dissociation rate constant (i.e., k_{d}, k_{off}). The dissociation rate constant is a mathematical constant describing the propensity of a larger object to separate (dissociate) reversibly into smaller components, as when a receptor/ligand complex dissociates into its component molecules. A third kinetic parameter of interest is the diffusion rate constant, k_{M}, which is a mathematical constant describing the rate at which labeled molecules diffuse toward a sensor. In addition, proteins or other molecules that are not involved in the binding interaction of interest can inhibit accurate measurement of such parameters.

### SUMMARY

Methods for quantitatively determining a binding kinetic parameter of a molecular binding interaction, for example where the determination involves a complex sample, are provided. Aspects of embodiments of the methods include:

producing a magnetic sensor device including a magnetic sensor in contact with an assay mixture including a complex sample including a magnetically labeled molecule to produce a detectable molecular binding interaction; obtaining a real-time signal from the magnetic sensor; and quantitatively determining a binding kinetics parameter of the molecular binding interaction from the real-time signal. Also provided are systems and kits configured for use in the methods.

### BRIEF DESCRIPTION OF THE FIGURES

FIG. 1 shows a schematic representation of antibody-antigen binding (not draft to scale), according to embodiments of the present disclosure.

FIG. 2 shows a schematic of sensor production and detection within the scope of embodiments of the present disclosure. Magnetic nanoparticles are used as labels.

FIG. 3 shows a schematic of embodiments wherein prey-protein coated MNPs are contacted with bait-protein coated sensors to produce a magnetic sensor.

FIG. 4 shows real-time data collected from a magnetic sensor for detection with antibody 5405 wherein the assay mixture included buffer, 50% plasma, and 80% plasma. Also shown are lines of best fit corresponding to the association and dissociation processes.

FIG. 5A shows real-time data collected with a conventional surface plasmon resonance (SPR) instrument with different concentrations of bovine serum albumin (BSA).

FIG. 5B shows an expanded view of a section of the real-time data shown in FIG. 5A. FIG. 6 shows real-time data collected from a magnetic sensor for detection with antibody 5405 in buffer with concentration of Tween 20, i.e. Polysorbate 20, of 0.05%, 0.5%, 1%, and 2%. Lines of best fit for the association and dissociation processes are also shown.

FIG. 7 shows Table 1 from Example 1.

FIG. 8 shows Table 2 from Example 2.

FIG. 9 shows Table 3 from Example 4.

### DETAILED DESCRIPTION

Methods for quantitatively determining a binding kinetic parameter of a molecular binding interaction, for example wherein the determination involves a complex sample, are provided. Aspects of embodiments of the methods include: producing a magnetic sensor device including a magnetic sensor in contact with an assay mixture including a complex sample including a magnetically labeled molecule to produce a detectable molecular binding interaction; obtaining a real-time signal from the magnetic sensor; and quantitatively determining a binding kinetics parameter of the molecular binding interaction from the real-time signal. Also provided are systems and kits configured for use in the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present invention and are disclosed herein just as if each and every such sub-combination of chemical groups was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing embodiments of the invention, aspects of embodiments of the methods will be described first in greater detail. Next, embodiments of systems and kits that may be used in practicing methods of invention are reviewed.

### Methods

As summarized above, embodiments of the invention are directed to methods of quantitatively determining a binding kinetic parameter of a molecular binding interaction of interest in a complex sample. The binding interaction of interest is, in certain embodiments, a binding interaction between a first and second molecule, e.g., between first and second biomolecules. For example, one of the first and second molecules may be a magnetically labeled molecule, and one of the first and second molecules may be a molecule that specifically binds to the magnetically labeled molecule. By “quantitatively determining” is meant expressing the binding kinetic parameter of interest in terms of a quantity, e.g., as a numerical value. By “binding kinetic parameter” is meant a measurable binding kinetic factor that at least partially defines a given molecular interaction and can be employed to define its behavior. Binding kinetic parameters of interest include, but are not limited to, an association rate constant (i.e., kₐ, kₒₙ), a dissociation rate constant (i.e., k_{d}, k_{off}), a diffusion-limited rate constant (i.e., k_{M}), an activation energy (i.e., E_{A}), transport parameters such as diffusivity, etc.

As summarized above, methods of the invention may include the following steps:
1) producing a magnetic sensor device in contact with an assay mixture that includes a magnetically labeled molecule;
2) obtaining a real-time signal from a magnetic sensor device; and
3) quantitatively determining a binding kinetic parameter of a molecular binding interaction from the real-time signal.

Each of these steps will now be described in greater detail.

Producing a Magnetic Sensor Device in Contact With an Assay Mixture that Includes a Magnetically Labeled Molecule

Aspects of the methods include producing a magnetic sensor device in contact with an assay mixture that includes a magnetically labeled molecule. The methods include producing a device or construct in which a magnetic sensor is contacted with a composition (e.g., an assay mixture) that includes the member molecules of a binding interaction of interest (i.e., the binding pair members of the binding interaction of interest) and a magnetic label, where the magnetic label may be a moiety or domain of one of the member molecules of the binding interaction of interest, or a component of a distinct molecule, e.g., a third molecule that specifically binds to one of the two member molecules of the binding interaction of interest. In the composition or assay mixture contacting the magnetic sensor, the magnetic label may be stably associated, e.g., either covalently or non-covalently, with one of the binding pair members to produce a magnetically labeled molecule. As will be further described below, the step of producing a magnetic sensor device in contact with an assay mixture that includes a magnetically labeled molecule may include a variety of different process subcombinations, e.g., in terms of when the binding pair members are contacted with each other, and or the magnetic sensor, the configuration of the binding pair members relative to the device, etc.

### Binding Pairs

A given binding interaction to be quantitatively kinetically analyzed according to methods as described herein may be made up of a binding pair of molecules, such as a first and second biomolecule. The binding pair of molecules may vary widely depending on the binding interaction of interest. Binding interactions of interest include any interaction between the binding pair of molecules, where the binding interaction occurs with specificity between the binding pair of molecules under the environmental conditions of the binding interaction. Examples of binding interactions of interest include, but are not limited to: nucleic acid hybridization interactions, protein-protein interactions, protein-nucleic acid interactions, enzyme-substrate interactions and receptor-ligand interactions, e.g., antibody-antigen interactions and receptor-agonist or antagonist interactions.

Examples of molecules that have molecular binding interactions of interest include, but are not limited to: biopolymers and small molecules, which may be organic or inorganic small molecules. A “biopolymer” is a polymer of one or more types of repeating units. Biopolymers may be found in biological systems (although they may be made synthetically) and may include peptides, polynucleotides, and polysaccharides, as well as such compounds composed of or containing amino acid analogs or non-amino acid groups, or nucleotide analogs or non-nucleotide groups. As such, biopolymers include polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. For example, a “biopolymer” may include DNA (including cDNA), RNA, oligonucleotides, and PNA and other polynucleotides as described in U.S. Pat. No. 5,948,902 and references cited therein. A “biomonomer” references a single unit, which can be linked with the same or other biomonomers to form a biopolymer (e.g., a single amino acid or nucleotide with two linking groups, one or both of which may have removable protecting groups).

The term “peptide” as used herein refers to any polymer compound produced by amide formation between an a-carboxyl group of one amino acid and an a-amino group of another group. The term “oligopeptide” as used herein refers to peptides with fewer than about 10 to 20 residues, i.e. amino acid monomeric units. The term “polypeptide” as used herein refers to peptides with more than 10 to 20 residues.

The term “protein” as used herein refers to polypeptides of specific sequence of more than about 50 residues and includes D and L forms, modified forms, etc. The terms “polypeptide” and “protein” may be used interchangeably.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Nucleic acids can be of any length, e.g., 2 bases or longer, 10 bases or longer, 100 bases or longer, 500 bases or longer, 1000 bases or longer, including 10,000 bases or longer. The term “polynucleotide” as used herein refers to single- or double-stranded polymers composed of nucleotide monomers of generally greater than about 100 nucleotides in length. Polynucleotides include single or multiple stranded configurations, where one or more of the strands may or may not be completely aligned with another. The terms “ribonucleic acid” and “RNA” as used herein mean a polymer composed of ribonucleotides. The terms “deoxyribonucleic acid” and “DNA” as used herein mean a polymer composed of deoxyribonucleotides. The term “oligonucleotide” as used herein denotes single-stranded nucleotide multimers of from about 10 to about 200 nucleotides in length, such as from about 25 to about 175 nucleotides in length, including from about 50 to about 160 nucleotides in length, e.g., 150 nucleotides in length.

In some instances, the binding pair of molecules are ligands and receptors, where a given receptor or ligand may or may not be a biopolymer. The term “ligand” as used herein refers to a moiety that is capable of covalently or otherwise chemically binding a compound of interest. Ligands may be naturally-occurring or manmade. Examples of ligands include, but are not restricted to, agonists and antagonists for cell membrane receptors, toxins and venoms, viral epitopes, hormones, opiates, steroids, peptides, enzyme substrates, cofactors, drugs, lectins, sugars, oligonucleotides, nucleic acids, oligosaccharides, proteins, and the like.

The term “receptor” as used herein is a moiety that has an affinity for a ligand. Receptors may be naturally-occurring or manmade. They may be employed in their unaltered state or as aggregates with other species. Receptors may be attached, covalently or noncovalently, to a binding member, either directly or via a specific binding substance. Examples of receptors include, but are not restricted to, antibodies, cell membrane receptors, monoclonal antibodies and antisera reactive with specific antigenic determinants, viruses, cells, drugs, polynucleotides, nucleic acids, peptides, cofactors, lectins, sugars, polysaccharides, cellular membranes, organelles, and the like. Receptors are sometimes referred to in the art as anti-ligands. As the term receptor is used herein, no difference in meaning is intended. A

“Ligand Receptor Pair” is formed when two molecules have combined through molecular recognition to form a complex.

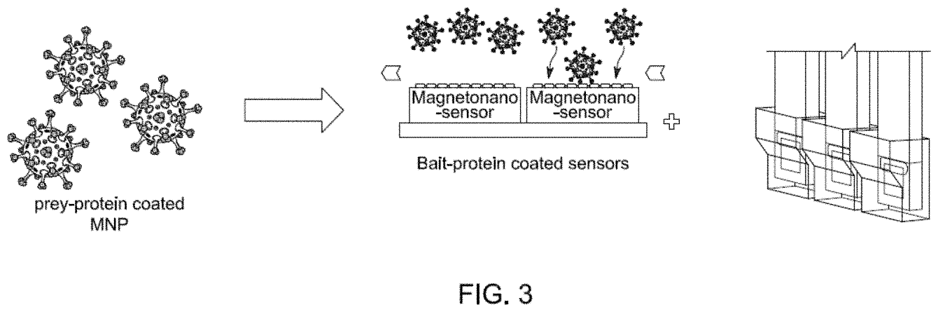

As shown in FIG. 3, magnetic nanoparticles (MNPs) can be coated with prey-protein and the magnetic sensor can be coated in bait-protein. The interaction between the prey and bait proteins can be the interaction that the binding kinetic parameters are determined for. In some cases, the prey protein can be a fully antibody. In other cases, the prey protein can be a fragment of the antibody.

In fact, various types of each binding member in a binding pair can be employed in the present methods. In some cases, the a first member of the binding pair is an antibody and a second member of the binding pair is a corresponding antigen. Such antibodies and antigens can be the full antibodies or antigens, e.g. as naturally occurring, or a fragment of an antibody or a fragment of an antigen can be used, or both. In some cases, the binding pair can include streptavidin and biotin.

### Magnetic Sensor Devices

Magnetic sensor devices of interest are those which generate an electrical signal in response to a magnetic label associating with a surface of the sensor. Magnetic sensor devices of interest include, but are not limited to, magnetoresistance sensor devices, including giant magnetoresistance (GMR) devices. GMR devices of interest include, but are not limited to spin valve detectors, and magnetic tunnel junction (MTJ) detectors.

### Spin-Valve Detectors

In some instances, the magnetic sensor is a spin valve detector. A spin valve detector is a metallic multilayer thin-film structure of two ferromagnetic layers spaced by a non-magnetic layer, e.g., copper. One ferromagnetic layer, called the pinned layer, has its magnetization pinned to a certain direction, while the magnetization of the other ferromagnetic layer, called the free layer, can rotate freely under an applied magnetic field. The electrical resistance of a spin valve depends on the relative orientation of magnetization of the free layer to that of the pinned layer.

When the two magnetizations are parallel, the resistance is the lowest; when antiparallel, the resistance is the highest. The relative change of resistance is called the magnetoresistance (MR) ratio. In some cases, the MR ratio of a spin valve can reach more than about 10% in a small magnetic field, e.g., about 100 Oe. Therefore, a spin valve can function as a sense element for the detection of magnetically labeled molecule associate with the sensor surface.

In certain embodiments, spin valves have a magnetoresistive (MR) ratio of about 1% to about 20%, such as about 3% to about 15 %), including about 5% to about 12%. Therefore, in certain embodiments, spin vales can detect a single magnetic label of about 10 nm size in a narrow bandwidth (i.e., about 1 Hz or less) or with lock-in detection. In these cases, by narrowing the noise bandwidth, a sufficient signal to noise ratio (SNR) is achieved even for single nanoparticle detection.

Spin valve detection may be performed with the in-plane mode (see e.g., Li, et al., J. Appl. Phys., vol. 93 (10): 7557 (2003)). In other embodiments, the vertical mode can be used when the electromagnetic interference (EMI) signal due to the AC tickling field in the detection system is detectable. The EMI signal tends to center at the frequency, f, of the AC tickling field, so it can be substantially eliminated or reduced by performing lock-in detection at the frequency 2f. Furthermore, in some instances, a 2-bridge circuit can be used to substantially remove the remaining EMI. Other signal acquisition and processing methods with an AC modulation sense current and an AC tickling field at two different frequencies may be used (e.g., S-J Han, H. Yu, B. Murmann, N. Pourmand, and S. X. Wang, IEEE International Solid-State Circuits Conference (ISSCC) Dig. Tech. Papers, San Francisco Marriott, Calif., USA, Feb. 11-15, 2007.)

In certain embodiments, the signal from the spin valve detector due to the magnetic label depends on the distance between the magnetic label and the free layer of the spin valve, in addition to the geometry and bias field of the spin valve itself. The detector voltage signal from a single magnetic label decreases with increasing distance from the center of the particle to the mid-plane of the spin valve free layer.

In certain embodiments, the free layer in the spin valve is on top of the pinned layer to facilitate detection of the magnetic label because the sensing magnetic field from a magnetic particle drops monotonically with the distance between the sensor and the particle. Minimization of the distance between the magnetic label and the top surface of the free layer, including the thickness of the passivation layer protecting the spin valve, may facilitate magnetic particle detection.

In certain embodiments, the spin-valve sensor may include a passivation layer on one or more of the detector surfaces. In some embodiments, the detector combines a thin (e.g., 60 nm or less, such as 50 nm or less, including 40 nm or less, 30 nm or less, 20 nm or less, or 10 nm or less) layer of passivation (e.g., in those embodiments where the detector is employed with magnetic nanoparticle tags with a mean diameter of 50 nm or less. In certain embodiments, larger, mircon-sized magnetic particles are employed. In some instances, the thin layers of passivation suitable for use with the presently disclosed detectors can have a thickness from about 1 nm to about 10 nm, such as from about 1 nm to about 5 nm, including from about 1 nm to about 3 nm. In certain embodiments, the thin layers of passivation suitable for use with the presently disclosed detectors can have a thickness from about 10 nm to about 50 nm, such as from about 20 nm to about 40 nm, including from about 25 nm to about 35 nm. The passivation layers may include, but are not limited to, Ta, Au, or oxides thereof, combinations thereof, and the like.

Further details regarding spin valve detectors and protocols for their use are provided in United States Patent Publication Nos. 2005/0100930 and 2009/0104707; the disclosures of which are herein incorporated by reference.

### Magnetic Tunnel Junction Detectors

In certain embodiments, the magnetic sensors are magnetic tunnel junction (MTJ) detectors. An MTJ detector is constructed similarly to a spin valve detector except that the non-magnetic spacer is replaced with an insulating layer (e.g., an insulating tunnel barrier), such as alumina or MgO, through which the sense current flows perpendicular to the film plane. Electron tunneling between two ferromagnetic electrodes is controlled by the relative magnetization of the two ferromagnetic electrodes, i.e., the tunneling current is high when they are parallel and low when antiparallel. In certain embodiments, the MTJ detector includes a bottom electrode, magnetic multilayers disposed on either side of the tunnel barrier, and a top electrode. In some cases, MTJ detectors have magnetoresistance ratios exceeding 200% (S. Ikeda, J. Hayakawa, Y. M. Lee, F. Matsukura, Y. Ohno,T. Hanyu, and H. Ohno, IEEE Transactions on Electron Devices, vol. 54, no. 5, 991-1001 (2007)) and large device resistances, yielding higher output voltage signals. In certain embodiments, the MTJ detector has a double-layer top electrode.

The first layer can be a metallic layer (e.g., gold layer) wherein the layer may have a thickness in some instances of 60 nm or less, such as 50 nm or less, including 40 nm or less, 30 nm or less, 20 nm or less, or 10 nm or less. The second layer can be a conductive metal, e.g., copper, aluminum, palladium, palladium alloys, palladium oxides, platinum, platinum alloys, platinum oxides, ruthenium, ruthenium alloys, ruthenium oxides, silver, silver alloys, silver oxides, tin, tin alloys, tin oxides, titanium, titanium alloys, titanium oxides, combinations thereof, and the like. In some instances, an aperture in the second layer is slightly smaller in size than the MTJ. In certain embodiments, the sensor is configured so that, during use, the distance between an associated magnetic label and the top surface of the free magnetic layer ranges from 5 nm to 100 nm, such as from 5 nm to 50 nm, including from 5 nm to 30 nm, such as from 5 nm to 20 nm, including from 5 nm to 10 nm. In some instances, this arrangement facilitates the reduction or substantial prevention of current crowding (see e.g., van de Veerdonk, R. J. M., et al., Appl. Phys. Lett., 71: 2839 (1997)) within the top electrode which may occur if only a thin gold electrode is used.

Except that the sense current flows perpendicular to the film plane, the MTJ detector can operate similarly to the spin valve detector, either with in-plane mode or vertical mode of the applied modulation field. As discussed above regarding spin valve detectors, in certain embodiments, the vertical mode of the applied modulation field can be used for reducing EMI and, similarly, thin passivation also applies to MTJ detectors. In addition, the first top electrode of thin gold on MTJ detectors can also facilitate electrical conduction, passivation, and specific biomolecular probe attachment.

In certain embodiments, at the same detector width and particle-detector distance, MTJ detectors can give larger signals than spin valve detectors. For example, for an MTJ detector with a junction area of 0.2 μm by 0.2 μm and resistance-area product of 1 kOhm-μm², operating with a MR of 250% at a bias voltage of 250 mV, and H_{b}=35 Oe, Hₜ=100 Oe rms, the voltage signal from a single 11 nm diameter Co nanoparticle whose center is 35 nm away from the midplane of the free layer may be about 200 μV. In some instances, this voltage is an order of magnitude, or more, greater than the voltage for similar-sized spin valve detectors.

Further details regarding MTJ detectors and protocols for their use are provided in United States Patent Publication Nos. 2005/0100930 and 2009/0104707, the disclosures of which are herein incorporated by reference.

### Magnetic Sensor Device Configurations

The magnetic sensor devices may have a variety of different configurations, e.g., with respect to sensor configuration, whether the devices are configured for batch or flow through use, etc. As such, any configuration that provides a magnetic sensor of the device to come into contact with a mixture of the binding members of the molecular binding interaction of interest and the magnetic label may be employed. Accordingly, configurations of the magnetic sensor device may include, but are not limited to: well configurations (in which the sensor is associated with the bottom or walls of a fluid containment structure, such as a well); flow through configurations, e.g., where the sensor is associated with a wall of a flow cell having a fluid input and output; etc.

In certain embodiments, the subject magnetic sensor device includes a substrate surface which displays two or more distinct magnetic sensors on the substrate surface. In certain embodiments, the magnetic sensor device includes a substrate surface with an array of magnetic sensors.

An “array” includes any two-dimensional or substantially two-dimensional (as well as a three-dimensional) arrangement of addressable regions, e.g., spatially addressable regions. An array is “addressable” when it has multiple sensors positioned at particular predetermined locations (i.e., “addresses”) on the array.

Array features (i.e., sensors) may be separated by intervening spaces. Any given substrate may carry one, two, four or more arrays disposed on a front surface of the substrate. Depending upon the use, any or all of the arrays may sense targets which are the same or different from one another and each may contain multiple distinct magnetic sensors. An array may contain one or more, including two or more, four or more, 8 or more, 10 or more, 50 or more, or 100 or more, 1000 or more, 10,000 or more, or 100,000 or more magnetic sensors. For example, 64 magnetic sensors can be arranged into an 8×8 array. In certain embodiments, the magnetic sensors can be arranged into an array with an area of 10 cm² or less, or 5 cm² or less, e.g., 1 cm² or less, including 50 mm² or less, 20 mm² or less, such as 10 mm² or less, or even smaller. For example, magnetic sensors may have dimensions in the range of 10 μm×10 μm to 200 μm×200 μm, including dimensions of 100 μm×100 μm or less, such as 90 μm×90 μm or less, for instance 50 μm×50 μm or less.

In certain embodiments, the magnetic sensor may include a plurality of linear magnetoresistive segments. For instance, the magnetic sensor can include 4 or more, such as 8 or more, including 12 or more, or 16 or more, e.g. 32 or more, for example 64 or more, or 72 or more, or 128 or more linear magnetoresistive segments. The magnetoresistive segments can each be 1000 nm wide or less, such as 750 nm wide or less, or 500 nm wide or less, for instance 250 nm wide or less. In some cases, the magnetoresistive segments can each be 50 nm thick or less, such as 40 nm thick or less, including 30 nm thick or less, or 20 nm thick or less, for example 10 nm thick or less. The magnetoresistive segments can each be 1000 nm long or less, or 750 nm long or less, or 500 nm long or less, or 250 nm long or less, for example 100 nm long or less, or 50 nm long or less.

The magnetoresistive segments may be connected together in series, or the magnetoresistive segments may be connected together in parallel. In certain instances, the magnetoresistive segments are connected together in series and in parallel. In these instances, two or more magnetoresistive segments may be connected together in parallel, and two or more groups of these parallel-connected magnetoresistive segments may be connected together in series.

In certain embodiments, at least some, or all, of the magnetic sensor or sensors of a given device have a binding pair member stably associated with a surface of the sensor. The binding pair member may vary, depending on the nature of the particular assay being performed. As such, the binding pair member may be a capture probe that specifically binds to a molecule of the molecular binding interaction of interest, or a molecule that participates in the molecular binding interaction of interest, e.g., a molecule that specifically binds to the magnetically labeled molecule. By “stably associated” is meant that the binding pair member and sensor surface maintain their position relative to each other in space for greater than a transient period of time under the conditions of use, e.g., under the assay conditions. As such, the binding pair member and sensor surface can be non-covalently or covalently stably associated with each other. Examples of non-covalent association include non-specific adsorption, binding based on electrostatic (e.g. ion, ion pair interactions), hydrophobic interactions, hydrogen bonding interactions, specific binding through a specific binding pair member covalently attached to the support surface, and the like. Examples of covalent binding include covalent bonds formed between binding pair member and a functional group present on the sensor surface, e.g. —OH, where the functional group may be naturally occurring or present as a member of an introduced linking group. Accordingly, the binding pair member may be adsorbed, physisorbed, chemisorbed, or covalently attached to the magnetic sensor surface.

Where a given device includes two or more magnetic sensors, each sensor may have the same or different binding pair member associated with its surface. Accordingly, different capture probes or molecules that bind to the magnetically labeled molecule may be present on the sensor surfaces of such devices, such that each magnetic sensor specifically binds to a distinct molecule. Such devices may also include sensors that are free of any binding pair member (e.g., where such blank sensors may serve as sources of reference or control electrical signals). In multi-sensor devices, areas in between the magnetic sensors may be present which do not carry any analyte specific probes. Such inter-sensor areas, when present, may be of various sizes and configurations. In some instances, these inter-sensor areas may be configured to reduce or prevent fluid movement among different sensors, e.g., where the inter-sensor areas include hydrophobic materials and/or fluid barriers (such as walls).

In certain embodiments, the substrate of the device, e.g., which may carry one or more arrays of distinct sensors, is shaped generally as a rectangular solid (although other shapes are possible), having a length of 1 mm or more and 150 mm or less, such as 1 mm or more and 100 mm or less, for instance 50 mm or less, or 10 mm or less; a width of 1 mm or more and 150 mm or less, such as 100 mm or less, including 50 mm or less, or 10 mm or less; and a thickness of 0.01 mm or more and 5.0 mm or less, such as 0.1 mm or more and 2 mm or less, including 0.2 mm or more and 1.5 mm or less, for instance 0.5 mm or more and 1.5 mm or less.

Electronic communication elements, e.g., conductive leads, may be present which are configured to electronically couple the sensor or sensors to “off-chip” components, such as device components, e.g., processors, displays, etc.

As described in greater detail below, a given magnetic sensor device may include a variety of components in addition to the sensor structure (e.g., array), such as described above. Additional device components may include, but are not limited to: signal processing components, data display components (e.g., graphical user interfaces); data input and output devices, power sources, fluid handling components, etc.

### Magnetic Labels

In embodiments of the methods, any convenient magnetic label may be employed. Magnetic labels are labeling moieties that, when sufficiently associated with a magnetic sensor, are detectable by the magnetic sensor and cause the magnetic sensor to output a signal. Magnetic labels of interest may be sufficiently associated with a magnetic sensor if the distance between the center of the label and the surface of the sensor is 200 nm or less, such as 100 nm or less, including 50 nm or less.

In certain embodiments, the magnetic labels are nanoparticles. Nanoparticles useful in the practice of certain embodiments are magnetic (e.g., ferromagnetic) colloidal materials and particles. The magnetic nanoparticles can be high moment magnetic nanoparticles which may be super-paramagnetic, or synthetic anti-ferromagnetic nanoparticles which include two or more layers of anti-ferromagnetically-coupled high moment ferromagnets. Both of these types of nanoparticles appear “nonmagnetic” in the absence of a magnetic field, and do not substantially agglomerate. In accordance with certain embodiments, magnetizable nanoparticles suitable for use include one or more materials such as, but not limited to, paramagnetic, super-paramagnetic, ferromagnetic, and ferri-magnetic materials, as well as combinations thereof.

In certain embodiments, the magnetic nanoparticles (also referred to as magnetic tags herein) have remnant magnetizations that are small, such that they will not agglomerate in solution. Examples of magnetic nanoparticles that have small remnant magnetizations include super-paramagnetic particles and anti-ferromagnetic particles. In certain cases, the magnetic tags have detectable magnetic moments under a magnetic field of about 100 Oe. In some instances, the size of the magnetic tags is comparable to the size of the target biomolecules so that the magnetic tags do not interfere with binding interactions between the molecules of interest. In certain embodiments, the magnetic tags are substantially uniform in shape and chemically stable in a biological environment, which may facilitate their use in the assay conditions. In some cases, the magnetic tags are biocompatible, i.e., water soluble and functionalized so that they may be readily attached to biomolecules of interest, e.g., a receptor that specifically binds to a target analyte.

In certain embodiments, the magnetic nanoparticles are high moment magnetic nanoparticles such as Co, Fe or CoFe nanocrystals, which may be super-paramagnetic at room temperature. The magnetic nanoparticles can be fabricated by chemical routes such as, but not limited to, salt reduction or compound decomposition in appropriate solutions. Examples of such magnetic nanoparticles include, but are not limited to, those described by S. Sun, and C. B. Murray, J. Appl. Phys., 85: 4325 (1999); C. B. Murray, et al., MRS Bulletin, 26: 985 (2001); and S. Sun, H. Zeng, D. B. Robinson, S. Raoux, P. M. Rice, S. X. Wang, and G. Li, J. Am. Chem. Soc., 126, 273-279 (2004).). In certain embodiments, the magnetic nanoparticles particles can be synthesized with controlled size (e.g., about 5-12 nm), are monodisperse, and are stabilized with oleic acid. Magnetic nanoparticles suitable for use herein include, but are not limited to, Co, Co alloys, ferrites, cobalt nitride, cobalt oxide, Co-Pd, Co-Pt, iron, iron alloys, Fe-Au, Fe-Cr, Fe-N, Fe3O4, Fe-Pd, Fe-Pt, Fe-Zr-Nb-B, Mn-N, Nd-Fe-B, Nd-Fe-B-Nb-Cu, Ni, Ni alloys, and the like. In some embodiments, a thin layer of gold is plated onto a magnetic core, or a poly-L-lysine coated glass surface can be attached to a magnetic core. Suitable nanoparticles are commercially available from, e.g., Nanoprobes, Inc. (Northbrook, Ill.), and Reade Advanced Materials (Providence, R.I.).

In some cases, magnetic nanoparticle tags are fabricated by physical methods (see e.g., W. Hu, R. J. Wilson, A. Koh, A. Fu, A. Z. Faranesh, C. M. Earhart, S. J. Osterfeld, S.-J. Han, L. Xu, S. Guccione, R. Sinclair, and S. X. Wang, Advanced Materials, 20, 1479-1483 (2008)) instead of chemical routes, and are suitable for labeling the target biomolecules to be detected. The magnetic tags may include two or more ferromagnetic layers, such as FeₓCo₁₋ₓ, where x is 0.5 to 0.7, or FeₓCo₁₋ₓ based alloys. In some cases, FeₓCo₁₋ₓ has a saturation magnetization of 24.5 kGauss. These ferromagnetic layers may be separated by nonmagnetic spacer layers such as Ru, Cr, Au, etc., or alloys thereof. In certain cases, the spacer layers include ferromagnetic layers coupled antiferromagnetically so that the net remnant magnetization of the resulting particles are zero or near zero. In certain embodiments, the antiferromagnetic coupling can be achieved via RKKY exchange interaction (see e.g., S. S. P. Parkin, et al., Phys. Rev. Lett., 64(19): 2304 (1990)) and magnetostatic interaction (J. C. Slonczewski, et al., IEEE Trans. Magn., 24(3): 2045 (1988)). In some cases, the antiferromagnetic coupling strength is such that the particles can be saturated (i.e., magnetization of all layers become parallel) by an external magnetic field of 100 Oe. In some cases, the antiferromagnetic coupling strength depends of the layer thicknesses and the alloy composition of the spacer layer.

In particular embodiments, to facilitate the bio-conjugation of the nanoparticle, a gold cap (or cap of functionally analogous or equivalent material) is layered on the top of the layers of anti-ferromagnetic material so that the nanoparticle can be conjugated to biomolecules via a gold-thiol or other convenient linkage. Surfactants may be applied to the nanoparticles, such that the nanoparticles may be water-soluble. The edges of the nanoparticles can also be passivated with Au or other inert layers for chemical stability.

Any convenient protocol may be employed to fabricate the nanoparticles described above. For instance, the layers of the nanoparticles can include nanometer-scale ferromagnetic and spacer layers deposited on substrates or release layers with substantially smooth surfaces. In some instances, a mask layer can be formed by imprinting, etching, self-assembly, etc. Subsequently, the mask layer and other unwanted layers may be removed and cleaned off thoroughly. Then, the release layer may be removed, lifting off nanoparticles which are the negative image of the mask layer. The particles may then be contacted with surfactants and biomolecules. In some cases, the substrate can be reused after thorough cleaning and chemical mechanical polishing (CMP).

In other embodiments, the nanoparticles are fabricated with a subtractive fabrication method. In this case, the layers are directly deposited on the release layer followed by a mask layer. The layers are etched through the mask layer, and eventually released from the substrate. These nanoparticles result from a positive image of the mask layer as opposed to the case in the additive fabrication method.

In certain embodiments, the size of the magnetic nanoparticles suitable for use with the present invention is comparable to the size of the biomolecules of the molecular binding interaction of interest, such that the nanoparticles do not interfere with the binding interaction of interest. Consequently, the size of the magnetic nanoparticles is, in some embodiments, sub-micron sized, e.g., from 5 nm to 250 nm (mean diameter), such as from 5 nm to 150 nm, including from 5 nm to 20 nm. For example, magnetic nanoparticles having a mean diameter of 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 55 nm, 60 nm, 70 nm, 80 nm, 90 nm, 100 nm, 110 nm, 120 nm, 130 nm, 140 nm, 150 nm, and 300 nm as well as nanoparticles having mean diameters in ranges between any two of these values, are suitable for use herein. Further, in addition to a spherical shape, magnetic nanoparticles suitable for use herein can be shaped as disks, rods, coils, fibers, and the like.

In some embodiments, the magnetic labels are colloidally stable, e.g., nanoparticle compositions may be present as a stable colloid. By colloidally stable is meant that the nanoparticles are evenly dispersed in solution, such that the nanoparticles do not substantially agglomerate. In certain embodiments, to prevent clumping, the nanoparticles may have no net magnetic moment (or a very small magnetic moment) in zero applied field. Anti-ferromagnetic particles may have zero magnetic moment in zero field at all sizes. In contrast, for a ferromagnetic particle, its size may be below the “super-paramagnetic limit”, which is, in some cases, about 20 nm or less, such as about 15 nm or less, including about 10 nm or less.

In certain embodiments, the synthetic nanoparticles can be produced in large quantities using a large wafer and standard vacuum thin film deposition processes. For example, with a 6-inch round wafer, 30-nm diameter nanoparticles at a rate of approximately 5×10¹² particles per run can be produced, assuming each particle occupies a square of 60 nm by 60 nm on the wafer.

In some instances, a molecule of a given binding interaction of interest and the magnetic label are stably associated with each other. By “stably associated” is meant that the biomolecule and the magnetic label maintain their position relative to each other in space for greater than a transient period of time under the conditions of use, e.g., under the assay conditions. As such, the biomolecule and magnetic label can be non-covalently or covalently stably associated with each other. Examples of non-covalent association include non-specific adsorption, binding based on electrostatic (e.g. ion, ion pair interactions), hydrophobic interactions, hydrogen bonding interactions, specific binding through a specific binding pair member covalently attached to the support surface, and the like. Examples of covalent binding include covalent bonds formed between the biomolecule and a functional group present on the surface of the label, e.g. —OH, where the functional group may be naturally occurring or present as a member of an introduced linking group.

### Assay Mixture Production

The magnetic sensor device which includes a magnetic sensor in contact with an assay mixture that includes a magnetically labeled molecule may be produced using any number of different protocols. In some cases, the assay mixture includes one or more complex samples, e.g. one complex sample. In some cases, the assay mixture includes one or more simple samples, e.g. a single simple sample and no complex samples.

### Complex Samples and Simple Samples

The sample that is contacted with the sensor surface may be a simple sample or complex sample. By “simple sample” is meant a sample that includes one or more members of the binding interaction and few, if any, other molecular species apart from the solvent. By “complex sample” is meant a sample that includes the one or more members of the binding interaction of interest and also includes many different proteins and other molecules that are not of interest. In certain embodiments, the complex sample assayed in the methods of the invention is one that includes 10 or more, such as 20 or more, including 100 or more, e.g., 10³ or more, 10⁴ or more (such as 15,000; 20,000 or even 25,000 or more) distinct (i.e., different) molecular entities that differ from each other in terms of molecular structure.

In certain embodiments, the complex sample is a blood sample. In some cases, the blood sample is whole blood. In some cases, the blood sample is a fraction of whole blood, e.g. serum or plasma.

In some cases, the complex solution is a non-blood fluid from an organism. In some cases, the non-blood fluid from an organism is cerebrospinal fluid (CSF), saliva, semen, vaginal fluid, lymph fluid, urine, tears, milk, or the external sections of the skin, respiratory tract, intestinal tract, or genitourinary tracts.

In some cases, the complex sample is a tissue sample. In some cases, the tissue sample is derived from a tumor. In some cases, the tissue sample is derived from non-tumorous tissue. In some cases, the complex sample is cell culture, or a part of a cell culture. In some cases, the cell culture or tissue sample is of a human or animal.

The complex sample can originate from any organism, including but not limited to a human, primate, monkey, fruit fly, rat, mouse, pig, or dog.

In some cases, the complex sample is whole blood, blood plasma, or blood serum of a human, mouse, rat, pig, dog, or monkey. In some cases, the complex sample is cerebrospinal fluid, saliva, or urine of a human, mouse, rat, pig, dog, or monkey.

In some cases, the complex sample includes components that are not of interest at concentrations sufficient to inhibit the accurate measurement of binding kinetic parameters with conventional methods. For example, in some cases, the inhibitory components of the complex mixture may inhibit accurately determining such parameters with surface plasmon resonance (SPR), whereas such parameters can be determined with relative accuracy with the present magnetic sensor methods. Several manners can be used to assess how accurately each method determines the binding kinetic parameters. Such manners can include whether the derivative of the smoothed real-time data has a single change in sign or multiple changes in sign. In other cases, such manners can include whether a discontinuity exists in the real-time data.

The assay mixture can include various amounts of a complex sample, for example, by mass the amount of a complex sample in the assay mixture can be 0.1% or more, such as 1% or more, 2% or more, 5% or more, 10% or more, 25% or more, 50% or more, 75% or more, 80% or more, 90% or more, 95% or more, 98% or more, or 100%. In some cases, the amount of the complex sample in the assay mixture is between 0.1% and 98%, such as between 1% and 95%, between 5% and 90%, or between 10% and 80%.

### Production of The Assay Mixture

The magnetic sensor device which includes a magnetic sensor in contact with an assay mixture that includes a magnetically labeled molecule may be produced using any number of different protocols. For example, a first molecule that specifically binds to the magnetically labeled molecule may be bound to a capture probe on the sensor surface, and then subsequently contacted with the magnetically labeled molecule (e.g., a second biomolecule which may be magnetically labeled). In these instances, methods may include providing a magnetic sensor device having a magnetic sensor which displays a capture probe that specifically binds to the first molecule, which also specifically binds to the magnetically labeled molecule; and then contacting the magnetic sensor with the first molecule and the magnetically labeled molecule. The contacting may include sequentially applying the first molecule, which binds to the surface and is capable of specific binding to the magnetically labeled molecule, and then applying the magnetically labeled molecule to the magnetic sensor.

Alternatively, the first molecule that specifically binds to the magnetically labeled molecule and the magnetically labeled molecule may be combined prior to contact with the sensor to form a complex, and the resultant complex may be allowed to bind to the capture probe on the sensor (e.g., where the binding kinetics of the binding interaction between the first molecule and the capture probe are of interest). In these instances, the contacting includes producing a reaction mixture that includes the first molecule that specifically binds to the magnetically labeled molecule and the magnetically labeled molecule, and then applying the reaction mixture to the magnetic sensor.

In yet other embodiments, the first molecule that specifically binds to the magnetically labeled molecule is first positioned on the sensor, and then contacted with the magnetically labeled second molecule. In these instances, the methods include providing a magnetic sensor device having a magnetic sensor which displays the first molecule (without an intervening capture probe); and then contacting the magnetic sensor with the magnetically labeled molecule.

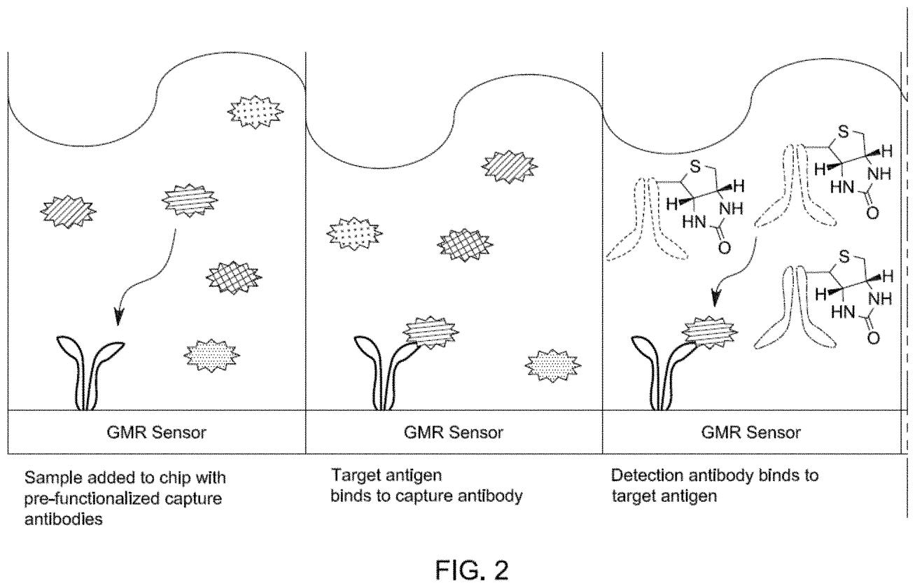

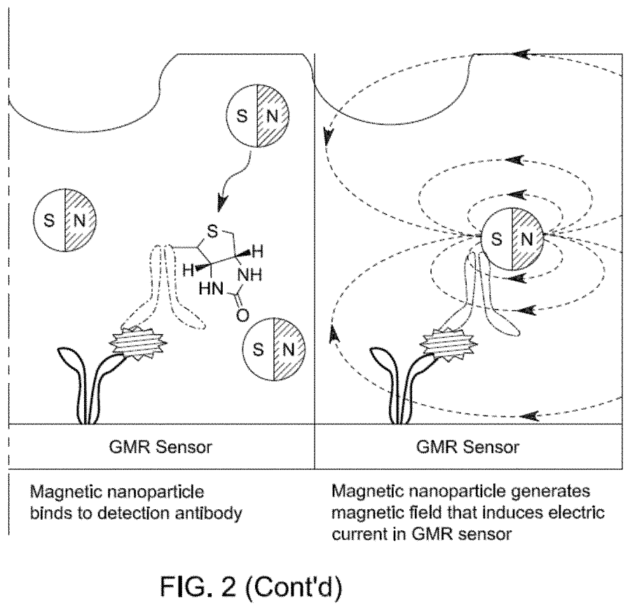

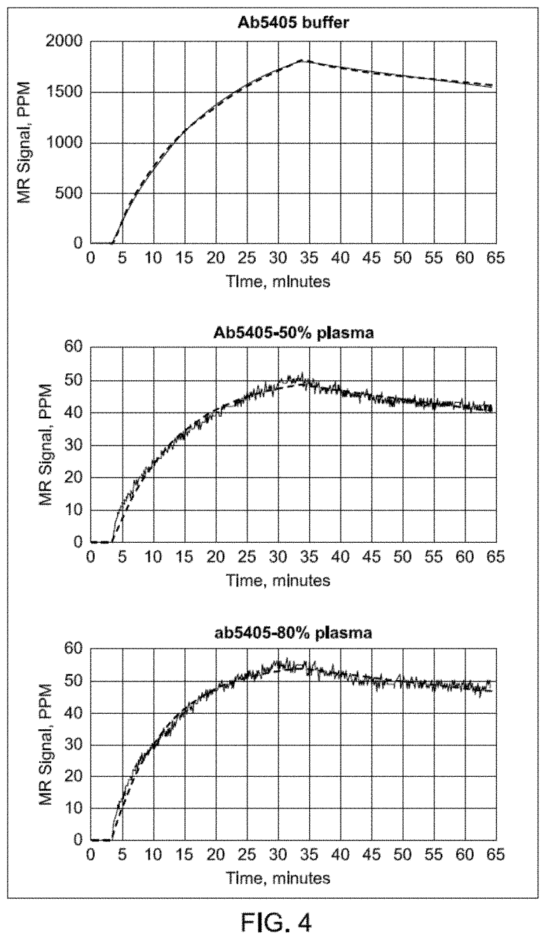

FIG. 4 provides an exemplary schematic illustrations for assay protocols that may be employed in the quantitative analysis of the binding kinetics of. In preparing the devices according to the protocol illustrated in FIG. 2, the binding kinetics of the interaction between the capture binding member (e.g., capture antibody or capture DNA) and the target member (e.g., analyte or target DNA) may be of interest. In such embodiments, the target and labeled member are contacted with each other first under binding conditions, and the resultant complex contacted with the sensor surface. Alternatively, in preparing the devices according to the protocols illustrated in FIG. 2, the binding kinetics of the interaction between the labeled binding member (e.g., labeled antibody or labeled DNA) and the target member (e.g., analyte or target DNA) may be of interest. In such embodiments, the target and capture member will be contacted with each other first under binding conditions, and the resultant sensor surface associated complex contacted with labeled member.

The contacting (including applying) steps described above are carried out under conditions in which the binding interaction of interest may occur. While the temperature of contact may vary, in some instances the temperature ranges from 1 to 95° C., such as 5 to 60° C. and including 20 to 40° C. The various components of the assay may be present in an aqueous medium, which may or may not include a number of additional components, e.g., salts, buffering agents, etc. In some instances, contact is carried out under stringent conditions. Stringent conditions may be characterized by temperatures ranging from 15 to 35° C., such as 20 to 30° C. less than the melting temperature of the probe target duplexes, which melting temperature is dependent on a number of parameters, e.g., temperature, buffer compositions, size of probes and targets, concentration of probes and targets, etc. As such, the temperature of hybridization may range from about 55 to 70° C., usually from about 60 to 68° C. In the presence of denaturing agents, the temperature may range from about 35 to 45, usually from about 37 to 42° C. The stringent hybridization conditions may be characterized by the presence of a hybridization buffer, where the buffer is characterized by one or more of the following characteristics: (a) having a high salt concentration, e.g. 3 to 6xSSC (or other salts with similar concentrations); (b) the presence of detergents, such as SDS (from 0.1 to 20%), triton X100 (from 0.01 to 1%), monidet NP40 (from 0.1 to 5%) etc.; (c) other additives, like EDTA (e.g., from 0.1 to 1 μM), tetramethylammonium chloride; (d) accelerating agents, e.g. PEG, dextran sulfate (from 5 to 10%), CTAB, SDS and the like; (e) denaturing agents, e.g. formamide, urea, etc.; and the like. Stringent conditions are conditions in which the stringency is at least as great as the specific conditions described above.

In some cases, the assay mixture can be a combination of a complex sample and one or more other components. In some cases, assay mixture can include a washing agent, a preservative, a buffer, a surfactant, an emulsifier, a detergent, a solubilizing agent, a lysing agent, water, a stabilizing agent, or a combination thereof. In some cases, the additional component is a surfactant. In some cases, the additional component is configured to inhibit non-selective binding of one or more elements within the complex mixture to the magnetic sensor. In some cases, the additional component is configured to increase the solubility of one or more components, e.g. proteins, within the complex mixture. In some cases, the preservative is a blood sample preservative. In some cases, the buffer is a bovine serum albumin (BSA) buffer.

The amount of the one or more additional components in the assay sample can be various amounts. For example, by mass the amount of each component in the assay mixture can be 0.1% or more by mass, such as 0.5% or more, 1% or more, 2% or more, 5% or more, 10% or more, 25% or more, 50% or more, 75% or more, 90% or more, or 95% or more.

In some cases, the assay mixture includes a blood sample and one or more of a buffer, a surfactant, and a preservative. In some cases, the assay mixture includes blood plasma, e.g. 10% or more of blood plasma, BSA buffer, and 0.1% or more of Polysorbate 20 surfactant. In some cases, the assay mixture includes blood serum, e.g. 10% or more of blood serum, BSA buffer, and 0.1% or more of Polysorbate 20 surfactant. In some cases, the assay mixture includes 10% or more of blood plasma or blood serum and BSA buffer. In some cases, the blood sample includes both blood plasma and blood serum. In some cases, the assay mixture includes a blood sample, a buffer, a surfactant, and a preservative. In some cases, the assay mixture includes a blood sample, a buffer, and a preservative. In some cases, the assay mixture includes a blood sample and a preservative and lacks buffer. In some of such cases, the assay mixture includes 50% or more by mass of the blood sample, e.g. 75% or more, 80% or more, 90% or more, or 95% or more.

In some cases, the complex solution includes a fraction of whole blood, e.g. serum or plasma, and the assay mixture also includes a surfactant. In some cases, the assay mixture further includes a buffer, e.g. BSA. In some cases, the assay mixture includes a fraction of whole blood and a preservative. In some cases, the assay mixture includes a fraction of whole blood, a buffer, a surfactant, and optionally a preservative.

In some cases, the surfactant is Polysorbate 20, also known as Tween 20 and polyoxyethylene (20) sorbitan monolaurate. In some cases, the surfactant is a nonionic surfactant. In some cases, the surfactant is Triton X-100, also known as polyethylene glycol p-(1,1,3,3-tetramethylbutyl)-phenyl ether. In some cases, the additional component is HAPS, DOC, NP-40, octyl thioglucoside, octyl glucoside or dodecyl maltoside. In some cases, the surfactant is a zwitterionic surfactant.

Obtaining a Real-Time Signal from a Magnetic Sensor

Following production of the device that includes the magnetic sensor in contact with an assay mixture (including the binding members of the binding interaction of interest and a magnetic label, e.g., as described above), aspects of the methods include obtaining a real-time signal from the magnetic sensor. As such, certain embodiments include obtaining a real-time signal from the device. Accordingly, the evolution in real time of the signal associated with the occurrence of the binding interaction of interest may be observed. The real-time signal is made up of two or more data points obtained over a given period of time of interest, where in certain embodiments the signal obtained is a continuous set of data points (e.g., in the form of a trace) obtained continuously over a given period of time of interest. The time period of interest may vary, ranging in some instances from1 second to 10 hours, such as 10 seconds to 1 hour and including 1 minute to 15 minutes. The number of data points in the signal may also vary, where in some instances, the number of data points is sufficient to provide a continuous stretch of data over the time course of the real-time signal.

In some embodiments, the signal is observed while the assay system is in the “wet” condition, that is, with a solution containing assay components (e.g., the binding members and magnetic label) still in contact with the sensor surface. As such, there is no need to wash away all of the non-binding or irrelevant molecules. This “wet” detection is possible because the magnetic field generated by the magnetic tag nanoparticle (e.g., with a diameter of 150 nm or less as described elsewhere) decreases rapidly as the distance from the nanoparticle increases. Therefore, the magnetic field at the sensor of the label bound to the captured binding members exceeds the magnetic field from the unbound magnetic labels in the solution, which are both at a greater distance from the detector and are in Brownian motion. The term “proximity detection” as used herein refers to this dominance at the sensor of the bound nanoparticles. Under the “proximity detection” scheme specifically bound magnetically labeled conjugates at the sensor surface can be quantified without washing off the nonspecific magnetic nanotags in the solution.

For a given binding interaction of interest, an assay may include obtaining a real-time signal for a single binding pair member concentration or multiple binding pair concentrations, such as 2 or more, 3 or more, 5 or more, 10 or more, 100 or more, or even 1,000 or more different concentrations. A given assay may contact the same sensor having the same capture probe concentration with multiple different binding pair member concentrations, or vice versa or a combination of different concentrations of capture probes and binding pair members, as desired.

As shown in FIG. 3, magnetic nanoparticles (MNPs) can be coated with prey-protein and the magnetic sensor can be coated in bait-protein. The interaction between the prey and bait proteins can be the interaction that the binding kinetic parameters are determined for.

In order to obtain real-time data that can be used to accurately determine such parameters, the absolute concentrations of the prey and bait proteins can be varied. In some cases, the absolute prey and bait concentrations can be adjusted to be sufficiently small so that the association and dissociation sections of the real-time signal can be fit with single-rate kinetic equations. Thus, adjusting the absolute concentrations of the prey and bait proteins can facilitate accurate determination of binding kinetic parameters. In addition, in some cases the relative amount of the prey proteins versus the bait proteins can be varied to facilitate fitting with single-rate kinetic equations and accurate determination of binding kinetic parameters. The real-time signals shown in FIGS. 4 and 6 were obtained with concentrations that facilitated fitting with single-rate kinetic equations.

Quantitatively Determining a Binding Kinetic Parameter from the Real-Time Signal

As summarized above, following obtainment of the real-time signal, the methods may include quantitatively determining a binding kinetic parameter of a molecular binding interaction from the real-time signal. In other words, the real-time signal is employed to quantitatively determine the binding kinetic parameters of interest, such that the binding kinetic parameters of interest are obtained from the real-time signal.

In some instances, the binding kinetic parameters of interest are quantitatively determined by processing the real-time signal with a fitting algorithm. By fitting algorithm is meant a set of rules that determines the binding kinetic parameters of interest by fitting equations to the real-time signal or signals obtained from a given assay, e.g., as described above. Any convenient fitting algorithm may be employed.

The binding kinetic parameters can be determined from the real-time signal in any suitable manner. In some cases, the parameters are determined, the values of kₒₙ, k_{off}, and K_{D} were calculated from the following equations:

Association Curve: Sₜ=S₀·[1−exp{−(c·kₒₙ+k_{off})·t}) (1)

Dissociation Curve: Sₜ=a·exp{−k_{off}·t) (2)

K_{D}=k_{off}/kₒₙ (3)

Using the presently described methods, accurate measurements of the binding kinetic parameters can be performed even when the assay mixture includes a complex sample solution. For example, even when the assay mixture includes 1% by mass or more of a complex sample solution, e.g. a blood sample, accurate measurements of the binding kinetic parameters can be performed.

In some cases, a kinetic binding parameter of a particular interaction has been measured, or can be measured, in another manner. For example, Surface Plasmon Resonance (SPR) with a simple solution, i.e. not a complex solution, might have been used to measure the kₒₙ of a particular interaction. However, the present methods allow for measurements of the same parameter with a complex sample solution-containing assay mixture and a magnetic sensor, e.g. a GMR sensor, such that good agreement between the previous value and the present value are obtained. Thus, the presence of the complex sample solution does not significantly negatively affect the accuracy of the measurement.

In some cases, the difference in kₒₙ values obtained from the present methods and a control method, e.g. SPR with a simple solution, 50-fold or less. For example, the present methods may result in an estimated kₒₙ value of 10⁴ M⁻¹, whereas the SPR with simple solution measurement may yield a value of 2×10³ M⁻¹, i.e. 5-fold less than the present method value. In some cases, the difference between the binding kinetic parameter determined from the real-time signal of the present methods and the binding kinetic parameter determined from a control method is 20-fold or less, such as 15-fold or less, 10-fold or less, 5-fold or less, 2-fold or less, 1-fold or less, 50% or less, or 25% or less. In some cases, such differences in parameters can be obtained even though the assay mixture includes 1% by mass or more of a complex solution, such as 5% or more, 10% or more, 25% or more, 75% or more, or 95% or more.

In some cases, the present methods do not include performing other such methods, e.g. SPR with a simple solution. In those cases, the parameter value obtained by the present methods is relative to the value obtained at another time, by another, or a combination thereof.

In some cases, usage of the method with a complex sample solution results in measured parameters that are within relatively good agreement with parameters with a simple solution. For example, the parameter obtained from measurement with a simple solution can be within 50-fold or less of a parameter obtained with a complex sample solution, such as 20-fold or less, such as 15-fold or less, 10-fold or less, 5-fold or less, 2-fold or less, 1-fold or less, 50% or less, or 25% or less. In some cases, such differences in parameters can be obtained even though one assay mixture includes less than 1% by mass, e.g. 0% by mass, of a complex sample whereas the other assay mixture includes 2% by mass or more of the complex sample, for example 5% or more, 10% or more, 25% or more, 75% or more, or 95% or more.

In some case, the accuracy and utility of the present methods is exemplified by generating real-time data that is suitable estimating the kinetic parameters. Thus, accuracy of the estimation can be increased by having data that accurately reflects the underlying interaction. In some cases, this accuracy exemplified when the measured GMR value increases for a time, reflecting association, followed by a decrease in the measured GMR value for a time, reflecting dissociation. For example, FIG. 4 show such a change in GMR value, as discussed in the Examples section. In such cases, the derivative of the real-time data has a single change in sign, e.g. the derivative is positive during the association phase and negative during the dissociation phase.

In addition, the real-time data can have temporary increases or decrease in the measured value that are attributable to, for example, statistical error. Thus, such errors are not considered in the assessment of, for example, the change in sign of the derivative. In fact, during data processing the real-time data can be subjected can be processed in a manner, e.g. smoothed, in order to reduce statistical noise and thereby increase the accuracy of the obtained parameter.

Hence, the accuracy of the present methods can be exemplified by smoothed real-time data that only has a single change in sign, e.g. corresponding to the association and dissociation phase.

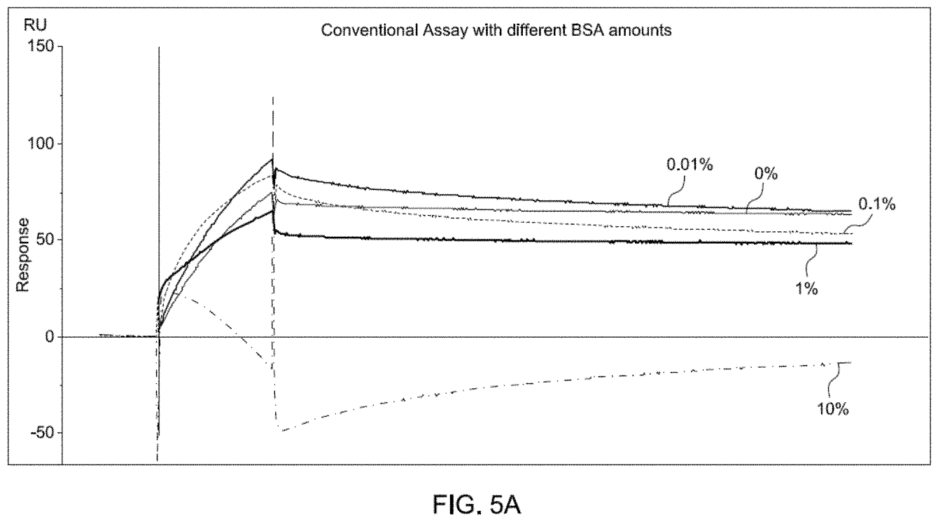

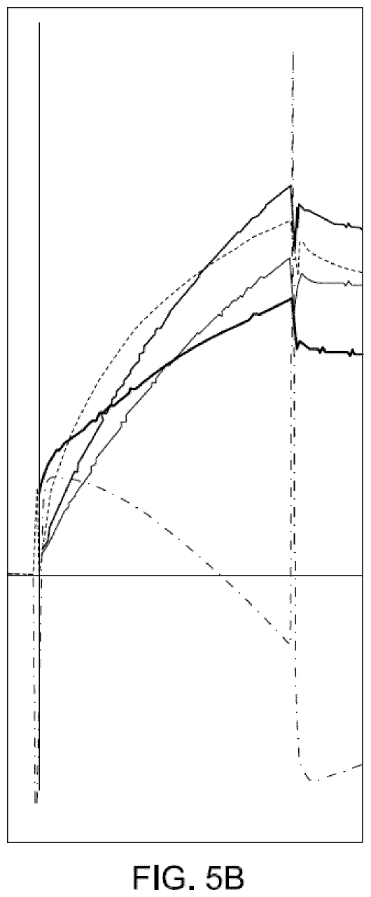

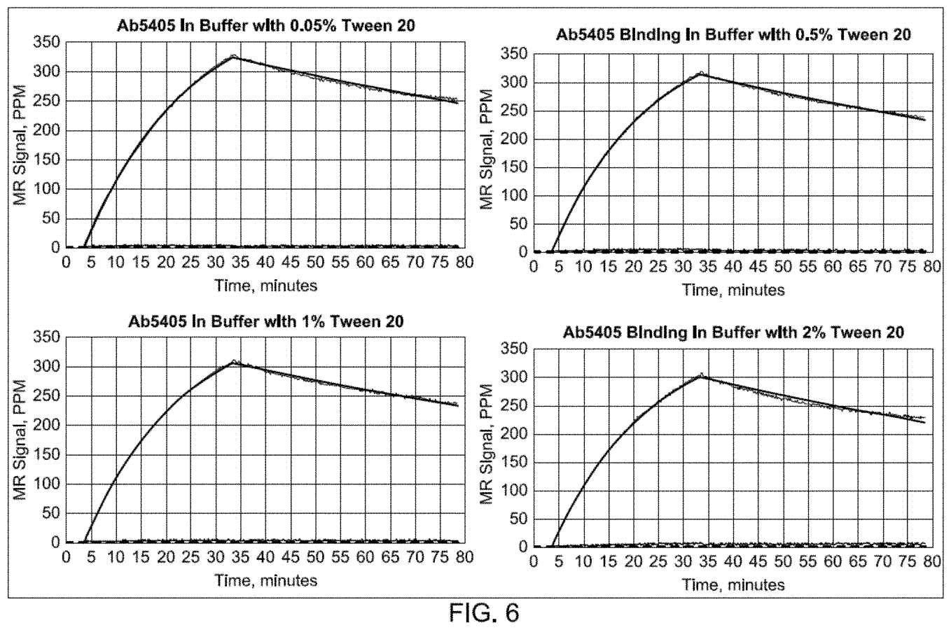

Similarly, the accuracy of the present methods can also be exemplified by the absence of a discontinuity in the data. Whereas various types of discontinuities can be present in real-time data, there are certain types of discontinuities that relate to the effect of complex sample solutions on the accuracy of obtaining accurate binding kinetic parameters. For example, as discussed in Example 3 below and shown in FIGS. 5A and 5B, the presence of the buffer BSA at certain concentrations caused a sharp increase, and then decreased, in the measured SPR signal. With the 10% BSA sample, this increase and decrease is shown as a sharp increase and decrease, wherein the curves approaching the sharp increase and decrease from the right and left do not trend towards the same value.

Although such discontinuities and errors can be classified in various manners, in some cases a discontinuity is located where the absolute value of the derivative of the smoothed real-time signal is 2 times or more than the average absolute value of the derivative of the smoothed real-time signal, such as 5 times or more, 10 times or more, 25 times or more, 50 times or more, or 100 times or more. For example, in FIG. 5A the absolute value of the derivative of the 10% BSA sample near the sharp-increase/sharp-decrease increases significantly, i.e. as shown by the sharp slope of the sharp-increase/sharp-decrease, compared with the gradual increase and gradual decrease, i.e. small derivative, of the curve elsewhere. In fact, as shown in FIG. 5B, even at lower concentrations of BSA, the real-time data shows a relatively abrupt change in derivative, indicating a discontinuity that negatively affects the ability to accurately obtain kinetic parameters from the data. As such, the present methods provide for accurate measurements of binding kinetic parameters by reducing or eliminating negative effects on the real-time data caused by components in the assay mixture that are not the components being studied, i.e. those containing complex sample solutions. For example, the present methods allow for accurate measurement of binding kinetic parameters even with 1% or more of a buffer or 10% or more of a blood sample. In contrast, other manners of attempting to measure such parameters with assay mixtures containing complex sample, i.e. SPR, result in erroneous and discontinuous data that provides inaccurate parameter estimations.

In some cases, the raw real-time data is smoothed before determining the binding parameters. In other cases, the raw real-time data is used to determine the binding parameters without being smoothed. In some cases, the method further includes smoothing the raw real-time data before performing the determining step. Manners of smoothing raw data are known in the art, and any suitable manner can be employed in the present methods.

In some instances, the real-time data can be analyzed and the binding kinetic parameters determined using fitting algorithms such as those described in U.S. Pat. No. 10,101,299 B2, the disclosure of which is incorporated by reference.

Where desired, the above quantitative determination protocol may be carried out with the aid of software and/or hardware configured to perform the above described protocol.

### Data Processing

The present methods provide for accurate quantitative determination of binding kinetic parameters, even when the assay mixture includes a complex sample. Such an advantage can be exemplified in various manners.

To illustrate such advantages, the real-time signal can be processed using mathematical methods, statistical methods, or a combination thereof that are known in the art. In some cases, such data processing can involve one or more of the operations of: taking an absolute value, taking a derivative, and smoothing the signal. When the data processing includes more than one of such steps, it is to be understood that such steps can be performed in any suitable order.

In some cases, the real-time signal is used to generate a derivative of the real-time data.

In some cases, the real-time signal is used to generate a smoothed derivative of the real-time signal by performing both a smoothing operation and taking a derivative. The smoothing operation can be performed first and followed by the taking a derivative operation, or the derivative can be taken first followed by smoothing.

In some cases, the real-time signal is used to generate an absolute value of the smoothed derivative of the real-time signal. Thus, such a procedure involves taking an absolute value, taking a derivative, and smoothing. Such operations can be performed in any suitable order. For example, the real-time signal can be used to generate a smoothed derivative of the real-time signal, and then the absolute value operation can be performed. In another example, the absolute value can be taken first, and then the derivative and smoothing operations can be performed in any order.

In some cases, the methods include such data processing steps. In other cases, the methods do not include such data processing steps, but rather the magnetic sensor device is configured such that if such data processing steps were performed then the resulting processed data would exemplify that the present methods, systems, and kits provide for accurate quantitative determination of binding kinetic parameters, even when the assay mixture includes a complex sample.

For example, in some cases the magnetic sensor device is configured such that if a smoothed derivative of the real-time signal was produced from the real-time signal, then the smoothed derivative of the real-time signal would contain only a single change in sign.

In other cases, the magnetic sensor is configured such that if the absolute value of the smoothed derivative of the real-time signal was produced from the real-time signal, then the smoothed real-time signal would contain a discontinuity where the absolute value of the smoothed derivative of the real-time signal is 5 times or more than the average absolute value of the smoothed derivative of the real-time signal.

In some cases, the methods include determining the binding kinetic parameters from a control, e.g. surface plasmon resonance (SPR). In such cases, the difference between the binding kinetic parameter determined from the real-time signal and the binding kinetic parameter determined from the control is 5-fold or less. In other cases, the methods do not include such determination with a control, but the magnetic sensor devices are configured such that the difference between the binding kinetic parameter determined from the real-time signal and the binding kinetic parameter determined from the control, e.g. wherein the value of the control parameter has previously been reported in scientific literature or was determined at another time, is 5-fold or less.

### Multiplex Analysis

Aspects of the invention include the multiplex analysis of two or more distinct binding interactions with the same sensor. By “multiplex analysis” is meant that two or more distinct binding interactions between different sets of binding molecules, in which the binding molecules and/or the magnetically labeled molecules are different from each other, e.g., by different sequence, are quantitatively analyzed. In some instances the number of sets is 2 or more, such as 4 or more, 6 or more, 8 or more, etc., up to 20 or more, e.g., 50 or more, including 100 or more, or 1000 or more, distinct sets. As such, in some cases, the magnetic sensor device may comprise two or more distinct magnetic sensors that each specifically detects a distinct binding interaction, such as 2 or more, or 4 or more, 6 or more, 8 or more, etc., up to 20 or more, e.g., 50 or more, including 100 or more, or 1000 or more, distinct magnetic sensors. In certain embodiments, of interest is the multiplex analysis of 2 to 1000 distinct binding interactions, such as 2 to 50, or 2 to 20 distinct binding interactions. Thus, in these embodiments, the magnetic sensor device may include 2 to 1000 distinct magnetic sensors that each specifically analyzes a distinct binding interaction, such as 4 to 1000 distinct magnetic sensors. In other cases, the magnetic sensor device may include 20 or less distinct magnetic sensors that each specifically analyzes a distinct binding interaction, such as 10 or less, including 4 or less distinct magnetic sensors.

### Devices and Systems

Aspects of the invention further include magnetic sensor devices and systems that are configured to quantitatively determine one or more binding kinetic parameters of a molecular binding interaction of interest. The devices and systems generally include a magnetic sensor; and a quantitative analysis module (e.g., processor) configured to receive a real-time signal from the magnetic sensor and quantitatively determine a binding kinetic parameter of a molecular binding interaction from the real-time signal. These two components may be integrated into the same article of manufacture as a single device, or distributed among two or more different devices (e.g., as a system) where the two or more different devices are in communication with each other, e.g., via a wired or wireless communication protocol.

Accordingly, aspects of the invention further include systems, e.g., computer based systems, which are configured to quantitatively assess binding interactions as described above. A “computer-based system” refers to the hardware means, software means, and data storage means used to analyze the information of the present invention. The minimum hardware of embodiments of the computer-based systems includes a central processing unit (CPU) (e.g., a processor), input means, output means, and data storage means. Any one of the currently available computer-based system may be suitable for use in the embodiments disclosed herein. The data storage means may include any manufacture including a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of an electronic controller, mainframe, server or personal computer (e.g., desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

Embodiments of the subject systems may include the following components: (a) a communications module for facilitating information transfer between the system and one or more users, e.g., via a user computer or workstation; and (b) a processing module for performing one or more tasks involved in the disclosed quantitative analysis methods.

In certain embodiments, a computer program product is described comprising a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein may be accomplished using any convenient method and techniques.

In addition to the sensor device and quantitative analysis module, systems and devices of the invention may include a number of additional components, such as data output devices, e.g., monitors, printers, and/or speakers, data input devices, e.g., interface ports, keyboards, etc., fluid handling components, power sources, etc.

### Utility

The subject methods, systems and kits find use in a variety of different applications where quantitative determination of a binding kinetic parameter of a binding interaction of interest is desired. In certain embodiments, the binding interaction is a binding interaction, such as, but not limited to, nucleic acid hybridization, a protein-protein interaction (e.g., as described in greater detail in the Experimental Section, below), a receptor-ligand interaction, an enzyme-substrate interaction, a protein-nucleic acid interaction, and the like.

In some instances, the subject methods, systems and kits find use in drug development protocols where the observation in real-time of molecular binding interactions may be desired. For example, drug development protocols may use the subject methods, systems and kits to monitor molecular the binding interactions between antibodies and antigens, or hybridization interactions between nucleic acids, or binding interactions between proteins, or binding interactions between receptors and ligands, or binding interactions between enzymes and substrates, or binding interactions between proteins and nucleic acids, and the like, in real time. For instance, CEA and VEGF are tumor markers and anti-VEGF antibody drugs, such as bevacizumab (Avastin; Genentech/Roche), are effective anti-cancer drugs. Another example is anti-EpCAM antibody, which has been formulated into a chemotherapeutic drug, edrecolomab. Monitoring binding interactions such as these may facilitate the development of other antibody-based drugs.

The subject methods, systems and kits also find use in analyzing molecular binding interactions between binding pairs that are included in complex samples. In some instances, the complex samples may be analyzed directly without separating the binding molecules of interest from the other proteins or molecules that are not of interest that may be in the sample. In certain cases, non-specific binding of proteins or molecules that are not of interest and unbound magnetic nanoparticles produce substantially no detectable signal in the subject methods, systems and kits. Thus, the subject methods, systems and kits find use in assay protocols where complex samples may be used and where the binding interactions of interest may be monitored in real-time with no washing of the sensor necessary for detection of the binding interactions of interest.

The real time binding assay and kinetic model disclosed herein may find use in applications such as epitope mapping. For example, the GMR sensor array has the ability to perform epitope mapping in a highly parallel fashion. Using capture antibodies, antigen can be selectively immobilized in a specific intra-molecular configuration on the sensor surface. The kinetic interaction of exposed epitopes on the captured antigen can be probed for affinity to various receptors or antibodies. For example, epidermal growth factor receptor (EGFR) is capable of binding EGF itself as well as proteins containing EGF-like repeats, such as EpCAM. By capturing proteins with EGF-like repeats using different monoclonal antibodies, and examining the binding of EGFR to these oriented proteins, an epitope map can be determined to evaluate the affinity of EGFR for various ligands containing EGF-like repeats. Using GMR sensors to probe exposed epitopes has applications ranging from massive screens of drug interactions with specific targets to parallel screening for specific domains of interest in the proteome.

The subject methods, systems and kits also find use in monitoring molecular binding interactions in both space and time. For example, the subject methods, systems and kits may be used to monitor localized cell-cell communication via cellular protein secretome analysis. By monitoring the diffusion of cellular protein secretions in space and time, the mechanisms of cell-cell communication may be determined.

The subject methods, systems and kits also find use in basic science research for understanding receptor-ligand binding interactions involved in signal transduction in cell biology or for profiling specific compounds of interest against an entire proteome. In addition, applications to clinical medicine are vast ranging from massive screens in directed protein evolution studies to investigating drug on-target and off-target cross-reaction binding kinetics.

The subject methods, systems and kits find use in such applications by allowing for determination of binding kinetic parameters when the assay mixture includes a complex sample.

### Computer Related Embodiments

Aspects of certain embodiments further include a variety of computer-related embodiments. Specifically, the data analysis methods described in the previous sections may be performed using a computer. Accordingly, embodiments provide a computer-based system for analyzing data produced using the above methods in order to provide quantitative determination of a binding kinetic parameter of a binding interaction of interest.

In certain embodiments, the methods are coded onto a computer-readable medium in the form of “programming”, where the term “computer readable medium” as used herein refers to any storage or transmission medium that participates in providing instructions and/or data to a computer for execution and/or processing. Examples of storage media include floppy disks, magnetic tape, CD-ROM, DVD, Blu-Ray, a hard disk drive, a ROM or integrated circuit, a magneto-optical disk, or a computer readable card such as a PCMCIA card or flash memory card, and the like, whether or not such devices are internal or external to the computer. A file containing information may be “stored” on computer readable medium, where “storing” means recording information such that it is accessible and retrievable at a later date by a computer. Of interest as media are non-transitory media, i.e., physical media in which the programming is associated with, such as recorded onto, a physical structure. Non-transitory media does not include electronic signals transmitted via a wireless protocol.

With respect to computer readable media, “permanent memory” refers to memory that is permanent. Permanent memory is not erased by termination of the electrical supply to a computer or processor. Computer hard-drive, CD-ROM, Blu-Ray, floppy disk and DVD are all examples of permanent memory. Random Access Memory (RAM) is an example of non-permanent memory. A file in permanent memory may be editable and re-writable.

### Kits

Also provided are kits for practicing one or more embodiments of the above-described methods. The subject kits may vary, and may include various devices and reagents. Reagents and devices of interest include those mentioned herein with respect to magnetic sensor devices or components thereof (such as a magnetic sensor array or chip), magnetic nanoparticles, binding agents, buffers, etc.

In some instances, the kits include at least reagents finding use in the methods (e.g., as described above); and a computer readable medium having a computer program stored thereon, wherein the computer program, when loaded into a computer, operates the computer to quantitatively determine a binding kinetic parameter of a binding interaction between the first and second molecules from a real-time signal obtained from a magnetic sensor; and a physical substrate having an address from which to obtain the computer program.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, Blu-Ray, etc., on which the information has been recorded. Yet another means that may be present is a website address which may be used via the Internet to access the information at a removed site. Any convenient means may be present in the kits.

The following examples are offered by way of illustration and not by way of limitation.

### EXPERIMENTAL

### General Methodology

A giant magnetoresistance (GMR) sensor array as described in Osterfield et al., Proc. Nat'l Acad. Sci USA (2008) 150:20637-206340 and Xu et al., Biosens. Bioelectron (2008) 24:99-103 was employed in the following general protocol:

Surface Functionalization: Sensor surfaces were functionalized to provide for stable association of a binding pair member, e.g., a capture antibody, first biomolecule, etc., onto the sensor surface. A cationic polymer such as polyethyleneimine (PEI) can be used to nonspecifically bind charged antibodies to the sensor surface via physisorption. Alternatively, a covalent chemistry can be used utilizing free amines on the antibody or free thiol groups. Additional details regarding surface functionalization for stable attachment of oligonucleotides is provided in Xu et al., Biosens. Bioelectron (2008) 24:99-103 and for antibodies is provided in Osterfield et al., Proc. Nat'l Acad. Sci USA (2008) 150:20637-206340. The binding pair member of interest was then contacted with the sensor surface to stably associate the binding member to the sensor surface.

Surface Blocking: Following surface functionalization and binding pair association, the sensor surface was blocked to prevent non-specific binding during the assay. In order to block the surface, a blocking buffer comprised of 1% BSA in PBS was added to the reaction well for one hour. Additional blocking protocols that may find use are described in Xu et al., Biosens. Bioelectron (2008) 24:99-103 and Osterfield et al., Proc. Nat'l Acad. Sci USA (2008) 150:20637-206340.

First Biomolecule: Following blocking, the sensor surface was contacted with a solution of the first biomolecule of interest, e.g., a purified solution of the first biomolecule or a complex sample that included the first biomolecule. For this step, a reaction well containing a solution of ˜1 nL-100 μL was used and the incubation time ranged from 5 minutes to 2 hours depending on the application.

Second Biomolecule: Following incubation, a solution containing the second biomolecule pre-labeled with the tag of interest (e.g., magnetic nanoparticle particle) was contacted with the sensor surface.

Monitoring Binding: Next, the binding kinetics of the second biomolecule to the first biomolecule were monitored and used to calculate binding rate constants based on the binding trajectory.

### GMR Sensors

The giant magnetoresistive (GMR) sensor used in the experiment had a bottom spin valve structure of the type: Si/Ta(5)/seed layer/IrMn(8)/CoFe(2)/Ru/(0.8)/CoFe(2)/Cu(2.3)/CoFe(1.5)/Ta(3), all numbers in parenthesis are in nanometers. Each chip contained an array of GMR sensors, which were connected to peripheral bonding pads by a 300 nm thick Ta/Au/Ta lead. To protect the sensors and leads from corrosion, two passivation layers were deposited by ion beam sputtering: first, a thin passivation layer of SiO₂(10 nm)/Si₃N₄(20 nm)/SiO₂(10 nm) was deposited above all sensors and leads, exposing only the bonding pad area; second a thick passivation layer of SiO₂(100 nm)/Si₃N₄(150 nm)/SiO₂(100 nm) was deposited on top of the reference sensors and leads, exposing the active sensors and bonding pad area. The magnetoresistive ratio was approximately 12% after patterning. The pinning direction of the spin valve was in-plane and perpendicular to the sensor strip. The easy axis of the free layer was set by the shape anisotropy to be parallel with the sensor strip. This configuration allowed the GMR sensors to work at the most sensitive region of their MR transfer curves.

Due to the GMR effect, the resistance of the sensor changed with the orientation of the magnetization of the two magnetic layers, which were separated by a copper spacer layer:

R(θ)=R₀−1/2δRₘₐₓ cos θ (10)

Here, R₀ is the resistance under zero magnetic field, δRₘₐₓ is the maximum resistance change and θ is the angle between the magnetization of the two magnetic layers. In the bottom spin valve structure, the magnetization of bottom magnetic layer (pinned layer) was pinned to a fixed direction, while the magnetic orientation of the top magnetic layer (free layer) was able to freely rotate with the external magnetic field. As a result, the stray field from the magnetic label can change the magnetization of the free layer and therefore change the resistance of the sensor.

Provided is a method for measuring binding kinetics with arrays of individually-addressable, magnetically-responsive nanosensors to simultaneously monitor the kinetics of numerous distinct proteins, binding to their corresponding targets, which are immobilized on a sensor surface. These magneto-nanosensors were successfully scaled to over 1,000 sensors per 1 mm² chip area. Analyte epitope mapping was demonstrated and spatial dynamics of protein diffusion in solution was visualized. In conjunction with these experiments, an analytical kinetics model which accurately describes the real-time binding of labeled proteins to surface-immobilized proteins was derived. The analytical model had close agreement to similar experiments using surface plasmon resonance and data from the literature. This model may be applied for antibody-antigen binding at sensitivities of 20 zeptomoles (20×10⁻²¹) of solute or less.

Soluble ligand was pre-labeled with a magnetic nanoparticle (MNP) in order to monitor the real-time binding kinetics of the ligand complex to antigen immobilized on the sensor surface. The magnetic field from the antibody-MNP complexes induced a change in electrical resistance in the underlying GMR sensor as the complexes were captured in real-time. Due to the rapid, real-time readout of the GMR sensor array, the kinetics of binding were monitored and quantified to determine the associated kinetic rate constants. The MNPs which label the protein or antibody of interest were twelve 10 nm iron oxide cores embedded in a dextran polymer , as determined by TEM analysis. The entire nanoparticle averaged 46±13 nm in diameter (from number weighted Dynamic Light Scattering). Based on the Stokes-Einstein relation, these particles had a translational diffusion coefficient of approximately 8.56×10⁻¹² m² s⁻¹. The MNPs had a zeta potential of −11 mV. These particles were superparamagnetic and colloidally stable, so they did not aggregate or precipitate during the reaction. In addition, the GMR sensors operated as proximity-based detectors of the dipole fields from the magnetic tags; thus, only tags within 150 nm of the sensor surface were detected. Therefore, unbound MNP tags contributed negligible signal in the absence of binding. Only bound magnetically labeled antibodies will be detected by the underlying GMR sensor, making this MNP-GMR nanosensor system useful for real-time kinetic analysis.

A GMR sensor array was fabricated with 1,008 sensors on a 1 mm² chip area. The calculated feature density was over 100,000 GMR sensors per cm². The sensor array was designed as a set of sub-arrays, where each sub-array occupied an area of 90 μm×90 μm. The sensor array was compatible with robotic spotters. Each sensor within a sub-array was individually addressable by row and column decoders via a shared 6-bit control bus fabricated with VLSI technology. The GMR sensor arrays allowed for parallel multiplex monitoring of protein binding kinetics.

### Magnetic Labels

The magnetic labels were obtained from Miltenyi Biotech Inc., referred to as “MACS” particles. Each MACS particle was a cluster of 10 nm Fe₂O₃ nanoparticles held together by a matrix of dextran. Due to the small size of the Fe₂O₃ nanoparticles, the MACS particle was superparamagnetic, with an overall diameter of 50 nm and contained 10% magnetic material (wt/wt). MACS particles were functionalized with the corresponding analyte being studied.

### Sensor Surface

The sensor surface was first rinsed with acetone, methanol and isopropanol. Subsequently, the sensors were exposed to oxygen plasma for three minutes. A 2% (w/v) polyallylamine solution in deionized water was applied to the sensor for 5 minutes. Other solutions may be used as desired, such as, but not limited to, solutions including anhydrise, poly allyl carboxylates, and the like. The chips were then rinsed with deionized water and baked at 150° C. for 45 minutes. For carboxylated surfaces, a 10% (w/v) solution of EDC and 10% (w/v) solution of NHS was then added to the sensor surface at room temperature for 1 hour.

### Kinetic Assay

After the sensor surface was functionalized with the appropriate capture protein, the GMR sensor array was placed in the test station and monitored in real time. The BSA blocking buffer was washed away and a 50 μL solution of the magnetically labeled detection antibody (made as described above) was added to the reaction well. The GMR sensor array was monitored over time as the magnetically labeled detection antibody bound to the corresponding protein. The binding curves, unique to each protein, could then be plotted and the binding rate constants could be determined. The assay was run for 5 minutes.

### Modeling and Fitting

Conventional pseudo-Langmuir curve fittings were applied to the real-time signals. As such, the values of kₒₙ, k_{off}, and K_{D} were calculated from the following equations:

Association Curve: Sₜ=S₀·[1−exp{−(c·kₒₙ+k_{off})·t}) (1)

Dissociation Curve: Sₜ=a·exp{−k_{off}·t) (2)

K_{D}=k_{off}/kₒₙ (3)

Fitting error is defined as the following: if N signal curves are measured from one chip, and curve j has nⱼ data points, and if D_{i,j} is denoted as the iₜₕ data point in curve j, and S_{i,j} as the ₜₕi data point in simulated curve j, then the fitting error for signal curve j is

$${E}_{j} = \sqrt{{\sum }_{i = 1}^{{n}_{j}} {( \frac{{S}_{i , j} - {D}_{i , j}}{{D}_{max , j}} )}^{2}} \qquad ( 4 )$$

where D_{max,j} is the maximum signal of signal curve . In this way, each experimental binding curve in the sensor array is compared to the binding curve predicted from the model. This error is then minimized to get the best fit and calculate kₒₙ. The absolute error was denominated by the maximum signal of the signal curve, so the fitting error was a percentage of the signal level. Therefore, percentage based relative fitting errors for large signal curves were similar to that of small signal curves. The total fitting error is:

E=\sqrt{Σⱼ₌₁^{N}Eⱼ²} (5)

This total fitting error is minimized in the fitting of the kinetic data presented herein.

### Example 1

### Measuring Binding Kinetic Parameters of Complex Samples with GMR Sensors

GMR sensors were used to measure binding kinetic parameters. Sensor surfaces were prepared by applying native human TSH proteins at different concentrations, from which an optimal condition (concentration) was selected for kinetic analysis.

Commercial TSH antibodies were individually conjugated to the magnetic nanoparticles (MNPs). Both the sensor surface and modified MNPs were blocked following conventional methods to prevent non-specific interactions.

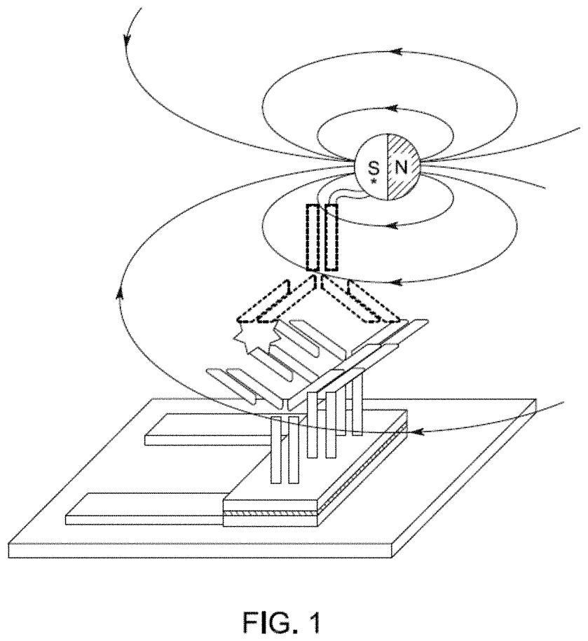

The real-time reading of the binding signals was realized by applying the modified MNPs to the sensors directly. Since only proximity signals are detected, they only reflect the specific binding of MNPs and the surface proteins. The mechanism of the interaction is shown in FIG. 1 and FIG. 2.

TSH protein and antibody interactions were studies wherein the assay mixture included: (i) simple solution with buffer but no blood sample; (ii) a complex solutions containing blood plasma, and (iii) buffers with different amount of the surfactant Tween 20, which is also known as Polysorbate 20. Up to 80% blood plasma and up to 2% Tween 20 were used. The two TSH antibodies of 5405 and 5409 were employed.

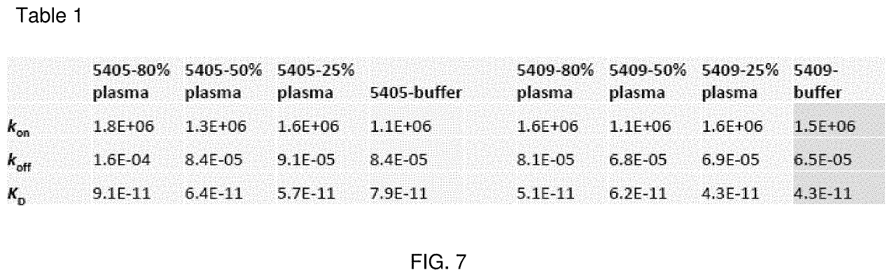

Binding studies with simple buffer, 25% blood plasma, 50% blood plasma, and 80% blood plasma were conducted. FIG. 4 show the results for the simple, 50%, and 80% samples. In each figure, the raw data and a kinetic best fit curve using equations (2)-(4) are shown. Values for kₒₙ, k_{off}, and K_{D} were calculated based on the best fit curves, yielding values that were varied less than 10-fold in all cases, as shown in Table 1 (see FIG. 7), even though the signals decreased with increasing plasma. Usually, the values for different samples, e.g. 80% plasma versus simple buffer, differed less than 1-fold, i.e. differed by less than 100%.

As shown in FIG. 4, the value of the smoothed real-time data increases for a time, i.e. from approximately 3 minutes until approximately 35 minutes, after which the value decreases. Thus, the derivative, i.e. slope, of the smoothed real-time data has a single change in sign. In particular, the derivative is positive between approximately 3 minutes and 35 minutes, and the derivative is negative after about 35 minutes. The interval from about 3 minutes to 35 minutes corresponds to the association process, i.e. kₒₙ, and the time after 35 minutes corresponds to the dissociation process, i.e. k_{off}. The lines of best fit shown in FIG. 4 correspond to fits obtained with the equations (2) and (3) discussed above. In addition, as most clearly shown in the 50% and 80% sample data of FIG. 4, the real-time data contains minor and temporary fluctuations in either a positive or negative direction that do not relate to the overall progression of the signal from an overall increase in value between about 3 minutes to about 35 minutes, followed by the decrease. Such minor and temporary fluctuations can be considered statistical noise, and the raw real-time data can be converted to smoothed real-time data by removing such minor and temporary fluctuations. Such manners of smoothing raw data are known in the art, and any suitable manner of smoothing the raw data can be employed.

### Example 2

### Comparing Parameters Obtained with Complex Samples and GMR Sensors to Literature Values

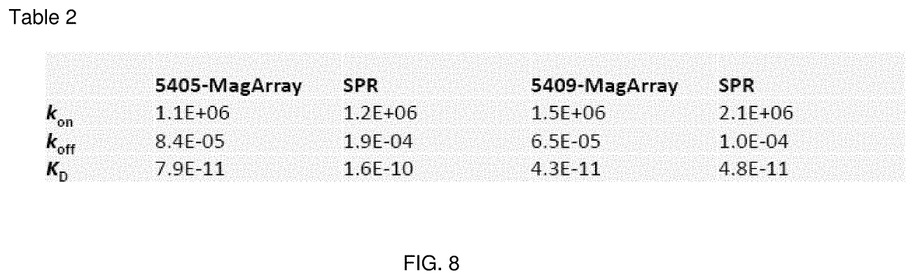

The binding kinetic parameters calculated in Example 1 have previously been measured using simple solutions and Surface Plasmon Resonance (SPR), i.e. the “literature values”. Table 2 (see FIG. 8) shows that the parameters calculated from the measurements of Example 1 were always within a 1-fold difference of the literature values, and usually significantly closer. Hence, the calculated parameters of Example 1 were in agreement with the literature values.

### Example 3

### Measuring Binding Kinetic Parameters of Complex Samples with SPR Sensors

Next, the same binding kinetic parameters of Example 1 were measured, but with the Biacore X100 instrument, which employs Surface Plasmon Resonance (SPR) instead of a GMR sensor. The same TSH proteins and antibodies were employed as in Example 1. The buffer was BSA at concentrations of 0%, 0.01%, 0.1%, 1%, and 10%.

However, as shown in FIGS. 5A and 5B, the measurement with the Biacore X100 instrument showed significant differences based upon the concentration of BSA. Thus, such significant differences using a showed that the presence of the

BSA buffer interfered with the accurate measurement of binding kinetic parameters when using an SPR instrument.

Such negative interferences from components other than the components of interest can be assessed in several manners. In some cases, the negative interferences will cause the derivative of the smoothed real-time data to have more than a single change in the sign. In fact, as shown in the FIG. 5B, i.e. an expanded view of a section of FIG. 5A, whereas the four lowest concentration samples always increased in signal until the sharp-increase/sharp-decrease, the 10% sample initially increased for a short period of time before decreasing. After the sharp-increase/sharp-decrease , the signal of the 10% sample once again increased.

Thus, even if the sharp-increase/sharp-decrease was not present in the 10% sample, the signal increased, decreased, and then increased again, yielding two changes in the sign of the derivative. In contrast, the data of FIG. 4 with the magnetic sensors of the present methods only had a single change in sign of the derivative.

Furthermore, each of the samples from the Biacrore X100 instrument shown in FIGS. 5A and 5B showed a momentary rapid change fluctuation in the measured signal, e.g. the sharp-increase/sharp-decrease of the 10% sample and rapid changes at the same time in the other samples. Thus, such changes add an extra two changes in sign of the derivative of the real-time data.

In addition, as shown clearly in the 10% sample of FIG. 5B, the signal shows a rapid increase and then decrease, before resuming a more gradual change in value. As such, the absolute value of the derivative of the smoothed 10% sample real-time data was significantly higher, i.e. greater than 5 times higher, than the absolute value of the derivative than the average absolute value of the derivative. Such a rapid change in value is considered herein to be an example of a discontinuity that exemplifies that the real-time data obtained with the Biacore X100 instrument under the tested conditions produced data with a low ability to produce accurate estimations of the binding kinetic parameters.

### Example 4

### Measuring Binding Kinetic Parameters of Complex Samples Containing a Surfactant with GMR Sensors

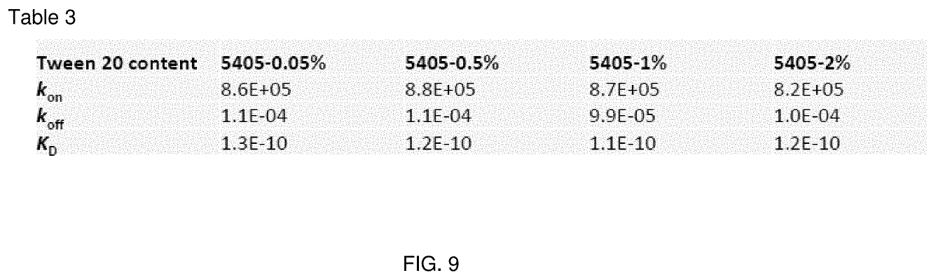

The effect of Polysorbate 20, a surfactant also known as Tween 20 and polyoxyethylene (20) sorbitan monolaurate, on measured binding kinetic parameters was investigated. Assay mixtures with 0.05%, 0.5%, 1%, and 2% of Polysorbate 20 were generated and measured with the 5405 antibody binding with TSH proteins. FIG. 6 shows the resulting raw data and lines of best fit, while Table 3 (see FIG. 9) shows the calculated binding kinetic parameters. As shown in FIG. 6, the derivative of the real-time data for each sample contains a single change in sign. In addition, the real-time data of FIG. 6 does not contain any rapid changes in value that would inhibit the ability to accurately calculate the binding parameters.

As such, it was found that consistent values of the parameters could be obtained even at Polysorbate 20 concentrations of up to at least 2%.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of quantitatively determining a binding kinetic parameter of a molecular binding interaction, the method comprising:
producing a magnetic sensor device comprising a magnetic sensor in contact with an assay mixture comprising 1% by mass or more of a complex sample comprising a magnetically labeled molecule to produce a detectable molecular binding interaction;
obtaining a real-time signal from the magnetic sensor; and
quantitatively determining a binding kinetic parameter of the molecular binding interaction from the real-time signal.

2. The method of claim 1, wherein the complex sample is a blood sample.

3. The method of claim 2, wherein the complex sample is whole blood.

4. The method of claim 2, wherein the blood sample is plasma.

5. The method of claim 2, wherein the blood sample is serum.

6. The method of claim 1, wherein the complex sample is a non-blood fluid from an organism.

7. The method of claim 6, wherein the non-blood fluid from an organism is cerebrospinal fluid, urine, or saliva.

8. The method of claim 1, wherein the complex sample is a cell culture or a tissue sample.

9. The method of claim 1, wherein the complex sample is obtained from or derived from a human, primate, monkey, fruit fly, rat, mouse, pig, or dog.

10. The method of claim 9, wherein the complex sample is obtained from or derived from a human.

11. The method of claim 1, wherein the assay mixture comprises 10% by mass or more of the complex sample.

12. The method of claim 11, wherein the assay mixture comprises 50% by mass or more of the complex sample.

13. The method of claim 12, wherein the assay mixture comprises 95% or more by mass of the complex sample.

14. The method of claim 1, wherein the assay mixture comprises one or more additional components selected from: a washing agent, a preservative, a buffer, a surfactant, an emulsifier, a detergent, a solubilizing agent, a lysing agent, and a stabilizing agent.

15. The method of claim 14, wherein the assay mixture comprises 0.1% by mass or more of the surfactant.

16. The method of 15, wherein assay mixture comprises 1% by mass or more of the surfactant.

17. The method of claim 14, wherein the surfactant is Polysorbate 20.

18. The method of claim 14, wherein assay mixture comprises a buffer.

19. The method of claim 18, wherein the buffer comprises bovine serum albumin.

20. The method of claim 1, wherein the difference between the binding kinetic parameter determined from the real-time signal and the binding kinetic parameter determined from a control is 20-fold or less.

21. The method of claims 20, wherein the control is determined by surface plasmon resonance.

22. The method of claim 20, wherein the difference between the binding kinetic parameter determined from the real-time signal and the binding kinetic parameter determined from the control is 5-fold or less.

23. The method of claim 22, wherein the difference between the binding kinetic parameter determined from the real-time signal and the binding kinetic parameter determined from the control is 2-fold or less.

24. The method of claim 1, further comprising:
producing a second magnetic sensor device comprising a magnetic sensor in contact with a second assay mixture comprising 1% by mass or less of the complex sample comprising the magnetically labeled molecule to produce the detectable molecular binding interaction;
obtaining a second real-time signal from the second magnetic sensor; and
quantitatively determining a second binding kinetic parameter of the molecular binding interaction from the second real-time signal,
wherein the difference between the binding kinetic parameter and the second binding kinetic parameter is 10-fold or less.

25. The method of claim 24, wherein the difference between the binding kinetic parameter and the second binding kinetic parameter is 2-fold or less.

26. The method of claim 1, further comprising producing a smoothed derivative of the real-time signal from the real-time signal.

27. The method of claim 26, wherein the smoothed derivative of the real-time signal contains only a single change in sign.

28. The method of claim 1, further comprising producing from the real-time signal an absolute value of the smoothed derivative of the real-time signal and a smoothed real-time signal.

29. The method of claims 28, wherein the smoothed real-time signal does not contain a discontinuity, wherein the discontinuity is located where the absolute value of the smoothed derivative of the real-time signal is 5 times or more than the average absolute value of the smoothed derivative of the real-time signal.

30. The method of claim 29, wherein the discontinuity is located where the absolute value of the smoothed derivative of the real-time signal is 25 times or more than the average absolute value of the smoothed derivative of the real-time signal.

31. The method of claim 30, wherein the discontinuity is located where the absolute value of the smoothed derivative real-time signal is 100 times or more than the average absolute value of the smoothed derivative of the real-time signal.

32. The method according to claim 1, wherein the binding kinetic parameter is an association rate constant (k_{d})

33. The method according to claim 1, wherein the binding kinetic parameter is a dissociation rate constant (k_{d}).

34. The method according to claim 1, wherein the binding kinetic parameter is a diffusion-limited rate constant (k_{M}).

35. The method according to claim 1, wherein the magnetic sensor comprises a molecule that is specifically bound to by the magnetically labeled molecule, and the producing comprises applying the magnetically labeled molecule to the magnetic sensor.

36. The method according to claim 1, wherein the magnetic sensor comprises a capture probe, wherein the capture probe and the magnetically labeled molecule each specifically bind to the molecule, and wherein the producing comprises sequentially applying the molecule and then the magnetically labeled molecule to the magnetic sensor.

37. The method according to claim 1, wherein the magnetic sensor comprises a capture probe, wherein the capture probe and the magnetically labeled molecule each specifically bind to a molecule, and the producing comprises producing a reaction mixture comprising the molecule and the magnetically labeled molecule and then applying the reaction mixture to the magnetic sensor.

38. The method according to claim 1, wherein the magnetic sensor is a spin valve sensor.

39. The method according to claim 1, wherein the magnetic sensor is a magnetic tunnel junction sensor.

40. A method of quantitatively determining a binding kinetic parameter of two or more distinct molecular binding interactions, wherein each distinct molecular binding interaction includes a different magnetically labeled molecule, the method comprising:
producing a magnetic sensor device comprising two or more distinct magnetic sensors each in contact with an assay mixture comprising 1% by mass or more of a complex sample comprising a magnetically labeled molecule to produce two or more distinct molecular binding interactions;
obtaining a real-time signal from each magnetic sensor; and
quantitatively determining a binding kinetic parameter for each of the two or more distinct molecular binding interactions from the real-time signal.

41. The method according to claim 40, wherein the binding kinetic parameter is an association rate constant (kₐ).

42. The method according to claim 40, wherein the binding kinetic parameter is a dissociation rate constant (kd).

43. The method according to claim 40, wherein the binding kinetic parameter is a diffusion-limited rate constant (km).

44. The method according to claim 40, wherein the binding interactions are binding interactions selected from the group consisting of nucleic acid hybridization interactions, protein-protein interactions, receptor-ligand interactions, enzyme-substrate interactions, and protein-nucleic acid interactions.
